(19) United States
(12) Patent Application Publication
(10) Pub. No.: US 2018/0131241 A1
(43) Pub. Date: May 10, 2018

(54) Device for transmitting energy and data and method for operating such device

(71) Applicant: Dualis MedTech GmbH, Seefeld (DE)

(72) Inventors: Heinz HORNUNG, Seefeld (DE); Stefan SCHWARZBACH, Wessling (DE); Christian HABERSETZER, Herrsching (DE); Dominik SCHUSTER, Wessling (DE); Soeren MICHEL, Seefeld (DE); Christoph SOMMER, SR., Starnberg (DE)

(21) Appl. No.: 15/647,728

(22) Filed: Jul. 12, 2017

(30) Foreign Application Priority Data
Jul. 12, 2016 (DE) 10 2016 212 626.1

(51) Int. Cl. H02J 50/80; H02J 50/10; A61F 2/08; A61M 1/12; A61N 1/362; A61N 1/372; A61N 1/378

(52) U.S. Cl. CPC H02J 50/80; H02J 50/10; A61F 2/08; A61M 1/122; A61N 1/362; A61F 2002/0894; A61N 1/3787; A61F 2250/0002; A61M 2205/3523; A61M 2205/8243; A61N 1/37229

(57) ABSTRACT

Energy transmission device for the wireless transmission of energy to an active implant, comprising: a transmitter coil adapted for electrical connection to an energy source, and an implantable receiver coil adapted for inductive coupling to the transmitter coil for wireless energy transmission, wherein an implantable primary coil is electrically connected to a modulator, the modulator modulating an AC voltage supplied to the implantable primary coil based on a data signal so that data transmission from the implantable primary coil to an extracorporeal secondary coil is performed, the frequency of the data transmission being different from the frequency at which the energy is transmitted from the transmitter coil to the receiver coil, and wherein information regarding the energy control of the energy to be transmitted from the transmitter coil is transmitted from the primary coil to the secondary coil by a pulse width modulated signal, other information not regarding the energy control is transmitted by means of a frequency modulation of the carrier frequency of the pulse width modulated signal or by a modulation of the frequency at which the pulses of the pulse width modulated signal are transmitted.

### BACKGROUND

### 1. Field of the Disclosure

The disclosure relates to an energy transmission device for the wireless transmission of energy to an active implant. The disclosure further relates to a method for operating such an energy transmission device.

### 2. Discussion of the Background Art

It is known from prior art to supply energy to active medical implants in a wireless manner. For this purpose, an extracorporeal transmitter coil is inductively coupled with an implanted receiver coil.

An inductive wireless transmission of energy often requires a transmission of data from the energy receiver to the energy transmitter. This data transmission may be used, for example, to transmit information for controlling the energy transmission or other information about the status of the receiver.

The control information is of particular importance if the relative position of the transmitter coil and the receiver coil cannot be determined exactly. This is often the case with medical implants, e.g. when a patient breathes. In such a situation the inductive coupling between the transmitter coil and the receiver coil changes so that the characteristics of the transmission path are not known exactly and may even change quickly. The control then has to adjust the parameters of the energy transmitter very frequently (e.g. ten or fifty times per second) based on the status information of the energy receiver.

With medical implants data transmission from the implant to the outside may even be critical under security aspects.

FIG. 1 illustrates the general functioning of a wireless energy transmission to an implant. A current supply is connected to an oscillator via which the transmitter coil L1 is supplied with AC voltage. The same induces an AC voltage in the receiver coil L2, which voltage is supplied to a rectifier. Thereby, the load, i.e. the implant, can be supplied with electric energy.

Various methods for a transmission of energy from the energy receiver to the energy transmitter are described in the following publications:
[1] Islam, Ashraf Bin: “Design of Wireless Power Transfer and Data Telemetry System for Biomedical Applications”, PhD Diss., University of Tennessee, 2011
[2] http://www.low-powerdesign.com/article_TI-Qi.html
[3] Wireless Power Consortium: “System Description Wireless Power Transfer”, Vol. I, Part 1, Version 1.1.2, June 2013
[4] https://en.wikipedia.org/wiki/Powermat_Technologies
[5] Rezence Alliance for Wireless Power: “A4WP Wireless Power Transfer System, Baseline System Specification (BSS)”, V 1.2.1, Final Approved Specification, May 7, 2014

The “Load Shift Keying (LSK) Method” is known from prior art, in which the load is changed on the receiver side in dependence on the data to be transmitted. The change of load can be effected using an additional resistive or capacitive load.

Publication [1] describes an LSK method in which the Load Shift Keying is effected using an additional resistive load.

Publications [2] and [3] describe a method in which the Load Shift Keying is effected using a capacitive load.

Another device for which a Load Shift Keying method is described is disclosed in publication [4].

The device in publication [5] uses a separate 2.4 GHz radio channel.

A separate radio channel increases the number of components required for the circuit and causes an increased susceptibility to failure. This is undesirable especially with medical implants. The necessary antenna additionally increases the structural space. A redundant design in security-critical applications may further require two different radio channels, which means more additional effort.

Owing to the principles involved, a separate radio channel may have a stronger interfering effect on other devices or may itself easily be subject to interferences.

As a matter of principle the LSK method is limited to data rates clearly below the frequency of the energy transmission. In particular when the transmission channel is difficult to determine, the load has to be changed rather drastically for an evaluable signal to arrive at the receiver. This, in turn, creates considerable losses which are particularly disadvantageous in a medical implant.

It is an object of the disclosure to provide an energy transmission device for the wireless transmission of energy and/or data from and/or to an active implant, which device has a simple structure and guarantees a reliable functioning. Further, it is an object of the disclosure to provide a method for operating such an energy transmission device.

### SUMMARY

The energy transmission device of the disclosure serves to wirelessly transmit energy to an active implant. An active implant is an implant that requires energy for its operation which is supplied from outside. This may e.g. be a cardiac pacemaker, a cardiac support device (ventricular assist device), artificial muscles etc.

The device of the disclosure comprises a transmitter coil for electrical connection to an energy source. The energy source may e.g. be a battery. The device further includes an implantable receiver coil adapted to be coupled with the transmitter coil for wireless energy transmission. An AC voltage is supplied to the transmitter coil, which voltage generates a varying magnetic field. The latter in turn induces an AC voltage in the receiver coil, which can be used to operate the active implant. This AC voltage can be transformed into DC voltage using a rectifier.

According to the disclosure an implanted primary coil of the device of the present disclosure is electrically connected to a modulator, an AC voltage supplied to the implanted primary coil being modulated by the modulator according to a data signal so that a data transmission occurs from the implanted primary coil to the extracorporeal secondary coil. The frequency of the data transmission differs from the frequency with which the energy is transmitted from the transmitter coil to the receiver coil.

According to the disclosure information regarding the energy control of the energy to be transmitted from the transmitter coil is transmitted from the primary coil to the secondary coil by a pulse-width modulated signal. In addition, other information that does not regard the energy control is transmitted by means of a frequency modulation of the carrier frequency of the pulse-width modulated signal or by a modulation of the frequency at which the pulses of the pulse-width modulated signal are transmitted.

Owing to the above-mentioned features it is possible to provide a simple and secure data transmission from the implant towards the external device. Based on the energy control information, the extracorporeal transmitter coil can control the power it supplies. This may be effected e.g. based on the duty cycle of the pulse-width modulated signal. Further, it is possible to transmit other information not regarding the energy control from the implanted device to the extracorporeal device without having to use an additional antenna or transmitter device for this purpose, which would go beyond the device used for energy control.

In a preferred embodiment the implanted primary coil is the receiver coil. In an addition or as an alternative, the extracorporeal secondary coil may be the transmitter coil. In other words: the already existing receiver coil may be used as the implanted primary coil and the already existing transmitter coil may be used as an extracorporeal secondary coil in order to provide the above-described communication channel for the energy control and the other information that do not regard the energy control.

In this embodiment the receiver coil, whose original function is to receive energy from the transmission coil, is used to transmit data to the transmitter coil. Thus, for data transmission purposes, no additional components are required for the actual signal transmission. Owing to the fact that the data transmission frequency differs from the energy transmission frequency, it can be ensured that the two transmission types do not influence each other. If a suitable frequency is used for data transmission, a high data rate can be guaranteed while, at the same time, the losses are low. As described further in the present application, electric components may be used for data transmission, which for the greater part are already present anyway.

The transmission of data according to the disclosure is preferably effected via the near field.

It is preferred that the modulator is inductively coupled to the implanted primary coil via a transformer. The transformer preferably is a transformer primary coil electrically connected to the modulator, and a transformer secondary coil electrically connected to the receiver coil.

It is preferred that the data transmission frequency is higher that the frequency at which the energy is transmitted from the transmitter coil to the receiver coil and that it is as far as possible from existing interferences, e.g. the harmonics of the energy transmission.

It is also possible to use a frequency for data transmission that is lower than the frequency for energy transmission. However, this is a less advantageous alternative because of the lower available data rate.

In a preferred embodiment a second transformer is provided for the inductive decoupling of the data signal received from the transmitter coil from an electric line connected to the extracorporeal secondary coil. This second transformer preferably comprises a transformer primary coil electrically connected to the extracorporeal secondary coil. The same is inductively coupled to a secondary coil which is electrically connected to an evaluation circuit.

The evaluation circuit has a band pass filter allowing only the useful frequency of the data transmission to pass. The band pass filter may comprise a prefilter that suppresses the energy transmission frequency to a degree sufficient to avoid a clipping of the main filter. The band pass filter may be passive or active (with amplification).

An amplifier may be connected downstream of the band pass filter, which amplifier raises the high-frequency data signal to a level suitable for demodulation. A demodulator may be arranged behind the amplifier, which demodulator extracts the data from the data signal.

In another preferred embodiment a second modulator is provided that is electrically connected to the extracorporeal secondary coil. The former serves to modulate an AC voltage supplied to the extracorporeal secondary coil in correspondence with a second data signal to be transmitted from the extracorporeal secondary coil to the implanted primary coil. Thereby it is possible, in addition to the transmission of a data signal from the implanted primary coil to the extracorporeal secondary coil, to transmit a data signal in the opposite direction, i.e. from the extracorporeal secondary coil to the implanted primary coil. For this purpose, no additional technical components except the above-mentioned modulator are required.

It is preferred that the frequency used for data transmission from the extracorporeal secondary coil to the implanted primary coil is different from the frequency used for data transmission from the implanted primary coil to the extracorporeal secondary coil. As an alternative, if the same frequency is used, transmission may be effected at different times in different directions. Further, as an alternative, different modulation methods could be used.

The disclosure further refers to a method for operating a device for the wireless transmission of energy to an active implant and of data from and/or to an active implant, in particular as described in the present application. The method of the present disclosure may comprise all features described in connection with the device of the present disclosure, and vice versa.

The method of the disclosure comprises the following method steps:
a) an AC voltage is supplied to a transmitter coil.
b) an implantable receiver coil is inductively coupled to the transmitter coil by arranging the two coils in proximity to each other. Here, it is preferred that both coils are arranged approximately congruently in the axial direction. Thereby, an AC voltage is induced in the receiver coil.

The method of the disclosure is characterized by the following steps:
c) an AC voltage supplied to the implanted primary coil is modulated in accordance to a data signal to be supplied from the implanted primary coil to the extracorporeal secondary coil.
d) the modulated AC voltage of the implanted primary coil induces a modulated AC voltage in the extracorporeal secondary coil.
e) a data signal is extracted from the modulated AC voltage and is evaluated. This may be effected by the above described evaluation circuit.
f) information regarding the energy control of the energy to be transmitted by the transmitter coil is transmitted from the primary coil to the secondary coil by a pulse width modulated signal.
g) further, other information that do not regard the energy control are transmitted by a frequency modulation of the transmission frequency of the pulse width modulated signal or by a modulation of the frequency with which the pulses of the pulse width modulated signals are transmitted.

However, steps a) and b) and steps c)-e) do not have to be performed at the same time. In other words: data transmission can be performed when no energy is transmitted via the coils at that moment.

The method of the present disclosure can also be use for data traffic in both directions.

In a preferred embodiment the signal strength of the data signal received by the extracorporeal secondary coil is measured so that, based thereon, the quality of the inductive coupling between the transmitter coil and the receiver coil is determined.

A preferred embodiment of the disclosure will be explained hereunder with reference to the drawings.

### BRIEF DESCRIPTION OF THE DRAWINGS

In the Figures:

FIG. 1 illustrates the basic functioning of a wireless energy transmission,

FIG. 2 shows an electric circuit diagram of an embodiment of the device according to the disclosure.

### DETAILED DESCRIPTION OF THE PREFERRED EMBODIMENT

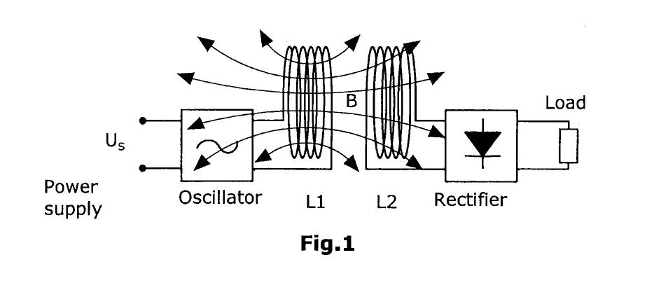

FIG. 1 has already been explained in the context of prior art.

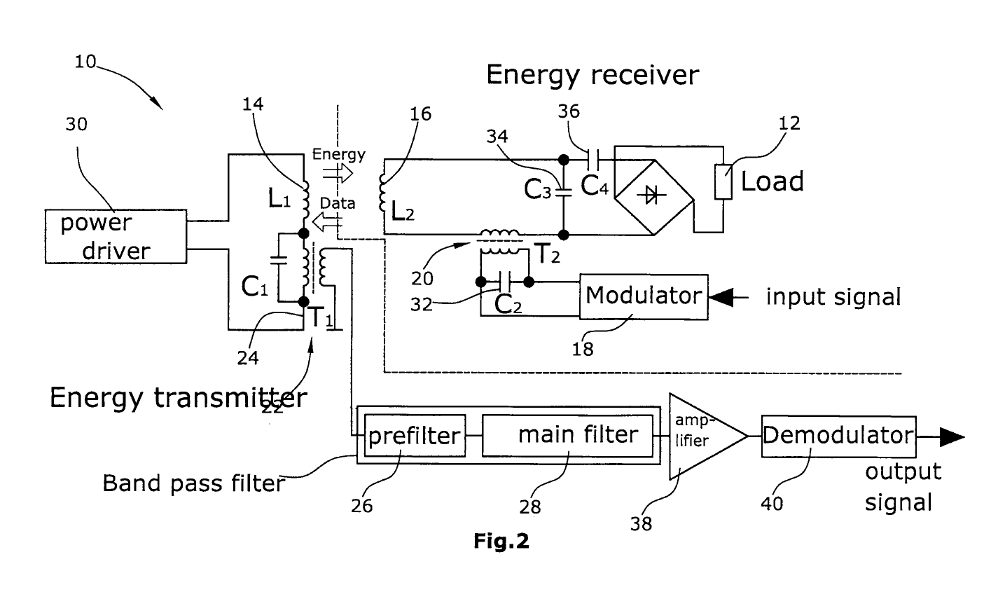

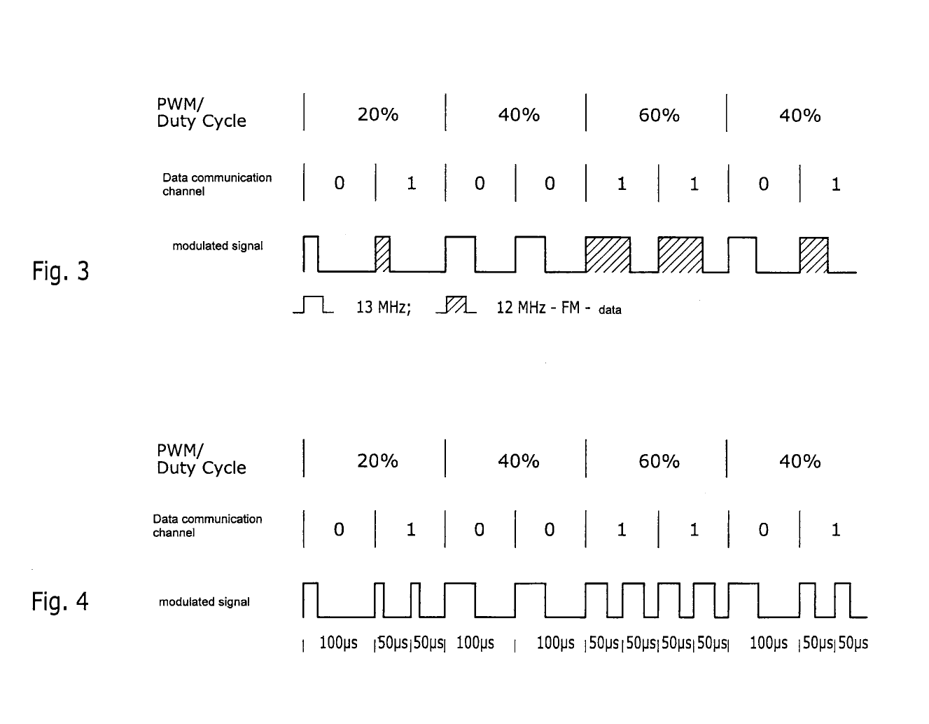

The extracorporeal coil 14 is illustrated on the left in FIG. 2. The coil is connected to a power driver 30 for coupling the energy in that is to be transmitted from the transmitter coil 14 to the implantable receiver coil 16.

On the side of the implant, the receiver coil 16 is illustrated on the right in FIG. 2. The same is connected to the first transformer 20 which comprises a primary coil and a secondary coil. Its primary coil is connected to the modulator 18. The same is used to modulate an AC voltage according to a data signal to be transmitted from the receiver coil 16 to the transmitter coil 14. The primary coil of this transformer may e.g. have an inductivity of about 1 μH.

A capacitor 32 is connected in parallel with the primary coil of the transformer 20 as a resonance capacitance, the capacitor forming a parallel resonant circuit together with the primary coil of the transformer 20, the circuit relieving the modulator.

34 and 36 denote tuning capacitors for energy transmission.

The receiver coil 16 transmits the data signal at a frequency clearly above the frequency for the transmission of energy. Thereby, it can be ensured that the harmonics of the energy transmission do not interfere with the data signal. For example, an energy transmission frequency of 100 kHz and a data signal frequency of 455 kHz can be used.

Information regarding the energy control of the energy to be transmitted by the transmitter coil 14 is transmitted from the receiver coil 16 to the transmitter coil 14 by a pulse width modulated signal. Further, other information that does not regard the energy control is transmitted using a frequency modulation of the carrier frequency of the pulse width modulated signal or a modulation of the frequency at which the pulses of the pulse width modulated signal are transmitted. In the former variant the carrier frequency can be modulated between 12 Megahertz and 13 Megahertz, wherein e.g. 12 Megahertz correspond to a logical 1 and 13 Megahertz correspond to a logical 0. This is illustrated in FIG. 3.

In the latter variant the frequency at which the pulses are transmitted is changed. Here, it is necessary that the two frequencies used for a logical 1 and a logical 0 are a multiple of each other. For example, the frequencies 10 kilohertz and 20 kilohertz may be used. This means that two pulses at 20 kilohertz correspond to a logical 1 and one pulse at 10 kilohertz corresponds to a logical 0. In order to still enable energy control via pulse width modulation in parallel with the above, the pulse width is adapted proportionally to the frequency (10 kilohertz or 20 kilohertz in the embodiment illustrated) so that the duty cycle of the pulse width modulated signal still remains the same. Thus, when a logical 0 is transmitted, only an electrical pulse with a frequency of 10 kilohertz is transmitted. The same has twice the pulse width of the two pulses for a logical 1 transmitted at a frequency of 20 kilohertz (assuming that the same duty cycle is to be transmitted in both cases). This embodiment is illustrated in FIG. 4.

The data signal transmitted by the receiver coil 16 is received by the transmitter coil 14 and is routed to the second transformer 22. The latter has a primary coil connected to the electric line 24 which, in turn, is connected to the transmitter coil 14. This primary coil may have a rather low inductivity of e.g. 1 μH and serves to decouple the high-frequency data signal. The same is then supplied to a prefilter 26 which preferably is a LC band pass filter. Thereby, the power frequency is limited prior to being supplied into the band pass filter.

The signal is then supplied to the band pass filter 28 which is a narrowband filter tuned to the data signal. The filter is preferably designed as a ceramic filter.

An amplifier 38 and a demodulator 40 are arranged downstream thereof.

The receiver coil 16 thus generates a magnetic field that corresponds to the data signal to be transmitted. The transmitter coil 14 picks up this magnetic field and generates a corresponding current which is coupled onto the evaluation circuit via the second transformer 22, the evaluation circuit comprising the prefilter 26, the band pass filter 28, the amplifier 38 and the modulator 40.

The output signal of the transformer 22 may at the same time be used to measure the primary current of the energy transmission.

Using a signal frequency of 455 kHz is particularly advantageous, because ceramic filters with very narrow bands are available for this frequency. Besides, it is of a sufficiently high frequency to provide a high data throughput.

The amplitude of the data signal may at the same time be used as a measure of the quality of the inductive coupling between the transmitter coil 14 and the receiver coil 16.

Basically, data transmission can also be performed from the transmitter coil 14 to the receiver coil 16. In this regard it is necessary to provide a corresponding modulator on the side of the transmitter coil 14, as well as the other components described, while on the implant side a corresponding evaluation circuit has to be provided. The corresponding circuit parts thus have to be switched.

Further, a bidirectional transmission is possible, wherein different frequencies are preferably used in this case.

It is further possible to use a plurality of frequencies in one direction and to thereby realize different data channels.

What is claimed is:

1. An energy transmission device for the wireless transmission of energy to an active implant, comprising
a transmitter coil adapted for electrical connection to an energy source, and
an implantable receiver coil adapted for inductive coupling to the transmitter coil for wireless energy transmission,
wherein
an implantable primary coil is electrically connected to a modulator, the modulator modulating an AC voltage supplied to the implantable primary coil based on a data signal so that data transmission from the implantable primary coil to an extracorporeal secondary coil is performed,
the frequency of the data transmission being different from the frequency at which the energy is transmitted from the transmitter coil to the receiver coil,
wherein
information regarding the energy control of the energy to be transmitted from the transmitter coil is transmitted from the primary coil to the secondary coil by a pulse width modulated signal,
other information not regarding the energy control is transmitted by a frequency modulation of the carrier frequency of the pulse width modulated signal or by a modulation of the frequency at which the pulses of the pulse width modulated signal are transmitted.

2. The energy transmission device of claim 1, wherein the carrier frequencies of the pulse width modulated signal range from 1 Megahertz to 13 Megahertz.

3. The energy transmission device claim 1, wherein the pulses of the pulse width modulated signal are transmitted at a frequency of between about 1 kilohertz to 20 kilohertz.

4. The energy transmission device of claim 1, wherein the other information is transmitted in a non-clocked manner, i.e. via an asynchronous communication channel.

5. The energy transmission device of claim 1, wherein the implanted primary coil is the receiver coil and/or the extracorporeal secondary coil is the transmitter coil.

6. The energy transmission device of claim 1, wherein the modulator is inductively coupled to an electric conductor via a transformer, the conductor being connected to the implanted primary coil.

7. The energy transmission device of claim 1, wherein the frequency of the data transmission from the implanted primary coil to the extracorporeal secondary coil is higher than the frequency at which the energy is transmitted from the transmitter coil to the receiver coil.

8. The energy transmission device of claim 1, further comprising a second transformer for an inductive decoupling of the data signal, which is received by the extracorporeal secondary coil, from an electric line connected to the extracorporeal secondary coil.

9. The energy transmission device of claim 1, further comprising a band pass filter allowing only the data transmission frequency to pass.

10. The energy transmission device of claim 1, further comprising a second modulator electrically connected to the extracorporeal secondary coil, for the modulation of an AC voltage supplied to the extracorporeal secondary coil on the basis of a second data signal to be transmitted from the extracorporeal secondary coil to the implanted primary coil.

11. The energy transmission device of claim 10, wherein the frequency used for the data transmission from the extracorporeal secondary coil to the implanted primary coil differs from the frequency used for the data transmission from the implanted primary coil to the extracorporeal secondary coil.

12. The energy transmission device of claim 10, wherein the modulation method used for the data transmission from the extracorporeal secondary coil to the implanted primary coil differs from the modulation method used for the data transmission from the implanted primary coil to the extracorporeal secondary coil.

13. A method for operating an energy transmission device for the wireless transmission of energy to an active implant, the method comprising the following steps:
a) supplying an AC voltage to a transmitter coil,
b) inductively coupling an implantable receiver coil to the transmitter coil so that an AC voltage is induced in the receiver coil,
c) modulating an AC voltage supplied to the implanted primary coil in accordance to a data signal to be supplied from the implanted primary coil to the extracorporeal secondary coil,
d) inducing a modulated AC voltage in the extracorporeal secondary coil by the modulated AC voltage of the implanted primary coil,
e) extracting and evaluating the data signal received by the extracorporeal secondary coil,
f) transmitting information regarding the energy control of the energy to be transmitted by the transmitter coil by a pulse width modulated signal from the implanted primary coil to the extracorporeal secondary coil,
g) transmitting other information that do not regard the energy control by a frequency modulation of the carrier frequency of the pulse width modulated signal or by a modulation of the frequency at which the pulses of the pulse width modulated signals are transmitted.

14. The method of claim 13, further comprising the following method step:
measuring the signal strength of the data signal received by the extracorporeal secondary coil, so that the quality of the inductive coupling between the transmission coil and the receiver coil is determined based thereon.

15. The method of claim 13, wherein in case of a modulation of the frequency at which the pulses of the pulse width modulated signals are transmitted, the pulse width is adjusted proportionally to the frequency at which the pulses of the pulse width modulated signal are transmitted.
